(19) United States
(12) Patent Application Publication
(10) Pub. No.: US 2023/0256207 A1
(43) Pub. Date: Aug. 17, 2023

(54) IV CATHETER DEVICE

(71) Applicant: New York Society for the Relief of the Ruptured and Crippled, maintaining the Hospital for Special S, New York, NY (US)

(72) Inventor: Michael Singleton, New York, NY (US)

(21) Appl. No.: 18/020,702

(22) PCT Filed: Aug. 9, 2021
(86) PCT No.: PCT/US2021/045187

§ 371 Date: Feb. 10, 2023

Related U.S. Application Data
(60) Provisional application No. 63/065,088, filed on Aug. 13, 2020.

(51) Int. Cl. A61M 25/06

(52) U.S. Cl. CPC A61M 25/0606; A61M 25/0693; A61M 25/0643

(57) ABSTRACT

An IV catheter device includes a needle component having an elongated needle with a sharp distal tip and a catheter component having a hollow body through which the needle passes. The IV catheter device further includes an actuatable obturator that is disposed within the needle component and moves between a pre-deployment position and a deployed position. In the deployed position, the actuatable obturator is disposed within an inner lumen of the elongated needle and a blunt distal end of the obturator is located distal to the sharp distal tip so as to blunt the elongated needle.

### CROSS REFERENCE TO RELATED APPLICATION

The present application claims priority to and the benefit of U.S. provisional patent application Ser. No. 63/065,088, filed Aug. 13, 2020, which is hereby expressly incorporated by reference in its entirety.

### TECHNICAL FIELD

The present invention is directed to intravenous equipment and more particularly, to IV catheter (delivery) systems that each includes an actuatable obturator that is configured to facilitate placement of the IV catheter system within the vein by blunting a sharp needle tip of the needle that is used to guide the delivery of the IV catheter system.

### BACKGROUND

As is well understood, IV stands for intravenous and concerns delivery fluids or medicines through a needle or tube (catheter) into a vein. The needle is usually placed in a vein near the elbow, the wrist or in the back of the hand. IV fluids or medicine are delivered in a wide array of different settings. One specific application in which medicine is delivered by an IV is the field of anesthesia which uses drugs or other methods to create a loss of awareness and block feelings of pain. It increases patient comfort and safety during medical procedures. Under general anesthesia, the patient is unaware and does not sense pain. Because the patient cannot breathe without help, a breathing machine is required. A breathing tube or other airway device delivers general anesthesia and maximizes patient safety. General anesthesia uses a variety of drugs and methods. The most common method is through breathing gas after an intravenous (IV) injection. The patient breathes in gases that are absorbed by the lungs and delivered through the bloodstream to the brain and spinal cord.

An IV catheter is the primary means for delivering the IV fluid/medicine to the patient. Intravenous (IV) cannulation is a technique in which a cannula (IV catheter) is placed inside a vein to provide venous access. Venous access allows sampling of blood, as well as administration of fluids, medications, parenteral nutrition, chemotherapy, and blood products.

Veins have a three-layered wall composed of an internal endothelium surrounded by a thin layer of muscle fibers that is surrounded by a layer of connective tissue. Venous valves encourage unidirectional flow of blood and prevent pooling of blood in the dependent portions of the extremities; they also can impede the passage of a catheter through and into a vein.

An IV catheter is introduced into the vein by a needle and then is fixed by being taped to a patient's skin. Most modern day IV catheters are equipped with a safety mechanism to shield the user from the needle as the needle is exposed and then later removed and retracted.

IV cannulation is typically performed by completing the following steps:
(1) a good vein is found to insert the IV catheter (e.g., a large vein located on the underside of the forearm);
(2) apply a tourniquet just a few inches above the site to get your veins to swell up for easy IV catheter insertion;
(3) disinfect the area with an alcohol pad. Wipe gently but thoroughly, ensuring an even coat of alcohol. This minimizes the chance of infection;
(4) use the right sized catheter. Ensure the needle doesn't touch anything other than the patient's skin. This can compromise their sterility and increase the risk of infection.
(5) stabilize the patient's limb with gentle pressure, taking care not to touch the IV site directly;
(6) remove the cap on the catheter and tightly pull the skin around the IV site. Insert the needle with minimum angle (as parallel to the skin as possible);
(7) the flashback of blood in the catheter's applicator will indicate that the vein has been hit, advance the needle one more centimeter (cm) into the vein;
(8) pull the needle back about 1 centimeter out of the vein. When the cannula is seated in the vein, remove the tourniquet and put a sterile bandage over the lower half of the catheter hub;
(9) dispose the needle for the safety of everyone involved and remove the tourniquet to prevent discomfort; and
(10) finally apply a piece of tape to the area around the catheter while attaching the IV tubing. Ensure there are no twists in the loop.

Unfortunately, there has been little improvement on the placement of an IV catheter over the years and most advancements have been in improvement in IV safety (prevention of needle sticks). There have been minimal design features to help novices achieve high rates of first attempt success when inserting and starting an IV. As mentioned, once the sharp tip of needle (beveled tip) enters vein, a flash of blood (a flashback) is seen in a chamber visible to the practitioner. However, when the tip of the needle enters the vein, the tip of catheter is still set back a short distance from the needle point. If a practitioner attempts to advance the catheter before the entire tip of the catheter is in the vein as well, it will not advance and will possibly disrupt and jeopardize the integrity of the vein. To prevent this from happening, once the flash is seen, the practitioner, must advance the needle further forward (a distance which depends on how big the needle is and how far the catheter tip is from the needle point) until the catheter is in the vein. This additional advancement runs the risk of “back-walling” the vein, or the needle point puncturing the other side of the vein and thus disrupting the integrity of the vein.

There is therefore a need for an IV catheter device that addresses and overcomes the above noted deficiencies and one that allows for proper placement of the catheter without the risk of “back-walling” the vein.

### SUMMARY

An IV catheter device according to one embodiment includes a needle component having an elongated needle with a sharp distal tip and a catheter component having a hollow body through which the needle passes. The IV catheter device further includes an actuatable obturator that is disposed within the needle component and moves between a pre-deployment position and a deployed position. In the deployed position, the actuatable obturator is disposed within an inner lumen of the elongated needle and a blunt distal end of the obturator is located distal to the sharp distal tip so as to blunt the elongated needle.

The present disclosure also sets forth a method for selectively blunting a sharp distal end of a needle that is part of an IV catheter device comprising the step of: actuating an obturator that is part of an IV catheter device to cause the obturator to travel within an inner lumen of the needle until a blunt distal end of the obturator is located distal to the sharp distal end of the needle so as to blunt the needle.

### BRIEF DESCRIPTION OF THE DRAWING FIGURES

FIG. 1 is an exploded perspective view of an IV catheter device;

FIG. 2 is a cross-sectional view of an IV catheter device with an obturator according to a first embodiment and being shown in a first position;

FIG. 3 is a cross-sectional view thereof with the device being shown in a second position;

FIG. 4 is a cross-sectional view thereof with the device being shown in a third position;

FIG. 5 is a cross-sectional view thereof with the device being shown in a fourth position;

FIG. 6 is a cross-sectional view of an IV catheter device with an obturator according to a second embodiment and being shown in a first position;

FIG. 7 is a cross-sectional view thereof with the device being shown in a second position;

FIG. 8 is a cross-sectional view of an IV catheter device with an obturator according to a third embodiment and being shown in a first position;

FIG. 9 is a cross-sectional view thereof with the device being shown in a second position;

FIG. 10A is cross-sectional view of an obturator having a first design;

FIG. 10B is cross-sectional view of an obturator having a second design;

FIG. 10C is cross-sectional view of an obturator having a third design;

FIG. 10D is cross-sectional view of an obturator having a fourth design;

FIG. 11 is a cross-sectional view of an IV catheter device with an obturator according to a fourth embodiment and being shown in a first position;

FIG. 12 is a cross-sectional view thereof with the device being shown in a second position;

FIG. 13 is a cross-sectional view of an IV catheter device with an obturator according to a fifth embodiment and being shown in a first position;

FIG. 14 is a cross-sectional view thereof with the device being shown in a second position;

FIG. 15 is a cross-sectional view of an IV catheter device with an obturator according to a sixth embodiment and being shown in a first position;

FIG. 16 is a cross-sectional view thereof with the device being shown in a second position;

FIG. 17 is a cross-sectional view of an IV catheter device with an obturator according to a seventh embodiment and being shown in a first position;

FIG. 18 is a cross-sectional view thereof with the device being shown in a second position;

FIG. 19 is a cross-sectional view of an IV catheter device with an obturator according to an eighth embodiment;

FIG. 20A is a cross-sectional view of one exemplary obturator with a first lock or retaining mechanism for maintaining the obturator in a deployed position;

FIG. 20B is a cross-sectional view of another exemplary obturator with a second lock or retaining mechanism for maintaining the obturator in a deployed position;

FIG. 20C is a cross-sectional view of another exemplary obturator with a third lock or retaining mechanism for maintaining the obturator in a deployed position;

FIG. 21 is a cross-sectional view of an IV catheter device with an obturator according to a ninth embodiment showing an echogenic obturator and being shown in a first position;

FIG. 22 is a cross-sectional view thereof with the device being shown in a second position; and

FIG. 23 is a cross-sectional view with the device being shown in a second position and showing another echogenic obturator design.

### DETAILED DESCRIPTION OF CERTAIN EMBODIMENTS

In accordance with the present disclosure, improved IV catheter devices are provided and are illustrated in the appended figures. It will be understood that components/elements that are used and present in multiple embodiments are numbered alike.

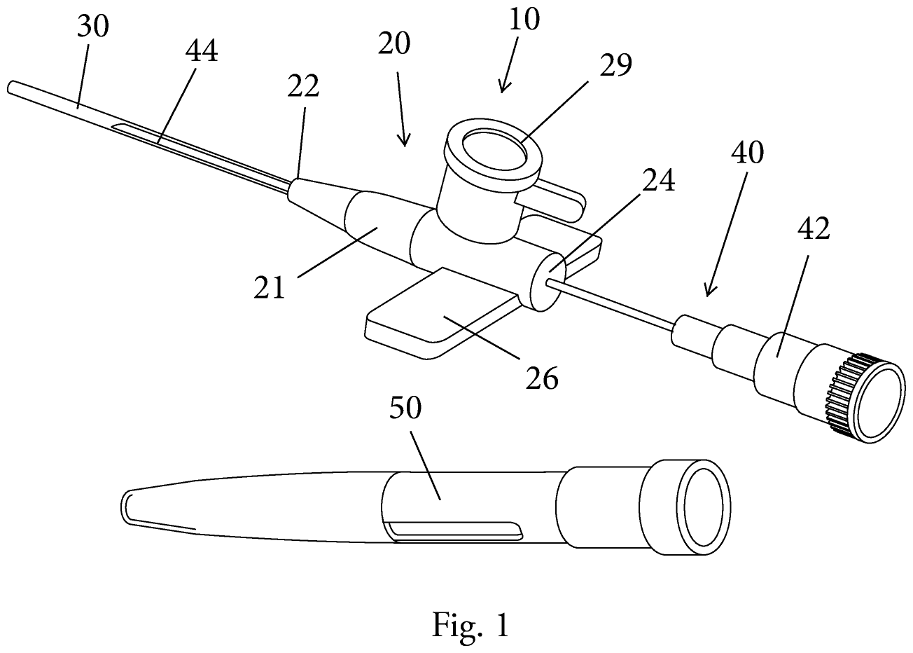

### IV Catheter Construction 10 (FIG. 1)

A traditional IV catheter is made up of a number of components that are assembled together to form an IV catheter device 10 as shown in FIG. 1. More specifically, the IV catheter device 10 typically includes an IV catheter component 20; a needle component 40, and a cover 50. The IV catheter component 20 has a hollow main catheter body 21 that has a first (distal) end 22 and an opposing second (proximal) end 24. The main catheter body 21 can have a pair of wings or side protrusions (tabs) 26 that extend radially outward from the main catheter body 21. The side protrusions 26 serve several different purposes in that they provide surfaces for grasping and manual handling of the IV catheter component 20 for advancing it into the vein. In addition, the side protrusions 26 provide surfaces for securing the IV catheter component 20 to the patient's body as by using an adhesive tape or the like. For example, each side protrusion 26 can have a piece of tape that is attached thereto and extends beyond edges for the side protrusion 26 for attaching to the skin.

At the distal end 22 of the main catheter body 21, an elongated catheter tip 30 is provided and extends distally from the distal end 22. The catheter tip 30 is a small hollow structure that has a center lumen formed therein and is integrally connected to the distal end 22 of the main catheter body 21. For example, the main catheter body 21 and the catheter tip 30 are typically formed as a single integral structure (e.g., single injection molded part). FIG. 1 shows an oversized catheter tip 30.

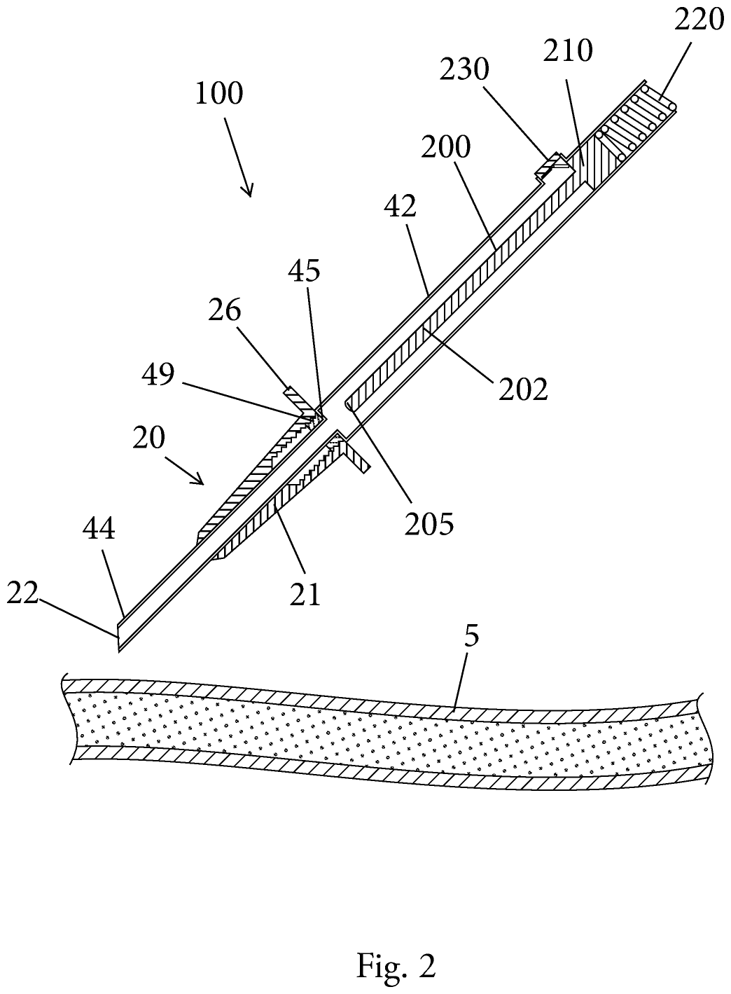

The elongated catheter tip 30 is the portion of the IV catheter component 20 that is driven into a vein 5 (FIG. 2), while the larger main catheter body 21 remains outside the patient's body in the embodiment of FIG. 1. This tip portion can also be referred to as a cannula. FIGS. 2-5 have smaller length tips compared to the more pronounced elongated catheter tip 30 and in fact, in FIGS. 2-5, the catheter tip comprises a tapered distal end of the main catheter body 21.

The IV catheter component 20 can include a valve 29 to allow for injection of fluid (e.g., drugs) with a syringe. Any number of suitable valves 29 can be used. Alternatively, the IV catheter component 20 can have a simple construction with no valve as shown in FIG. 2.

The proximal end 24 is the end which allows connection to an intravenous infusion line (tubing) and capping in between uses. As is known, the infusion line is placed in fluid communication with the main catheter body 21 for delivering fluid from a source, such as an IV bag or the like, to the vein through the catheter tip 30.

The needle component 40 serves as a guidewire for inserting the cannula (catheter tip 30) into the vein. As shown, the needle component 40 is a separate part from the IV catheter component 20. The needle component 40 includes a hub portion 42 for grasping the needle component 40 and includes of course, an elongated hollow needle 44. The hub portion 42 is much wider than the needle and an inner shoulder 45 (FIG. 2) is formed at the end of the hub portion 42 where the needle is coupled. The hub portion 42 can be a plastic structure. The hub portion 42 can also have a coupling structure 49 (such as a boss) (FIG. 2) at the proximal end of the needle 44. This coupling structure can be in the form of cylindrical boss with the needle entering the center thereof. As described herein, the proximal end of the IV catheter main body 21 can be detachably coupled to this boss for temporarily affixing the main catheter body 21 to the needle.

The needle 44 is usually formed of metal. As shown, the needle 44 is sized so that it is received within the hollow main catheter body 21 and travels into the hollow catheter tip 30. The needle 44 can be advanced beyond the distal tip.

Typically, the open distal end of the needle 44 is beveled to create a sharp end that can be advanced through the skin into the vein.

As described herein, the hub portion 42 can be also referred to as a flash chamber since one of the primary features of the hub portion 42 is to define and contain the flash chamber.

FIG. 2 shows a slightly different type of IV catheter component 20 in that it does not include a long elongated catheter tip 30 but instead the distal end 22 of the catheter body 21 is tapered and it is this distal end 22 that is inserted into the vein through the skin that has been pierced by the needle 44.

The needle component 40 can also include a structure that serves to assist in retention of the IV catheter component 20 and more specifically, a boss 49 or the like can be provided and is sized to establish a friction fit between the IV catheter component 20 and the needle and in particular, the boss 49 as shown in FIG. 2. In other words, the open proximal end of the IV catheter component 20 is inserted over the boss 49 and a friction fit is established to hold and retain the IV catheter component 20 in place.

Blood in a person's veins is under a higher than atmospheric pressure and therefore, when the IV needle 44 is placed in a vein, the higher pressure forces blood to travel through the needle 44. The blood can flow because the other end of the needle, called the flash chamber, that is located within the hub portion 42, has a fiber filter or the like in it that allows air to escape. After the catheter component 20 is advanced into the vein, the needle component 40 is removed and discarded.

The cover or protection cap 50 is designed to fit over the catheter tip 30 and shield the needle that is located inside the catheter tip 30. Before use, the cover 50 is removed and discarded.

### Actuatable Obturator

As described herein, in accordance with the present disclosure, the actuatable obturator functions to selectively transform the sharp distal tip of the needle 44 into a blunt end after the needle is inserted into the vein.

### Solid Obturator 200

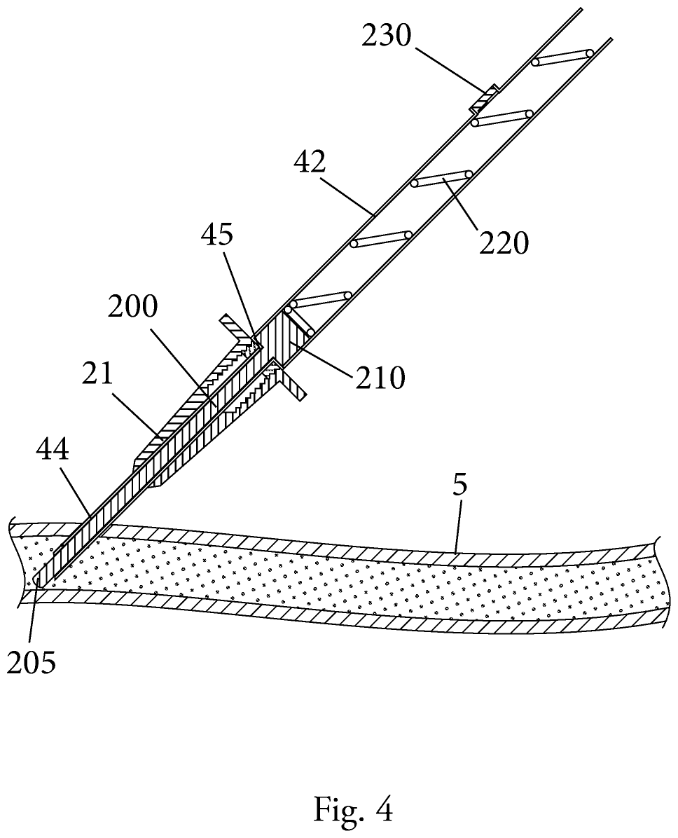

In accordance with the present disclosure, an actuatable obturator 200 is provided as part of an IV catheter device 100 for selectively transforming the sharp distal tip of the needle 44 into a blunt end (See, FIG. 4). As is known, an obturator is an instrument that blocks up an opening in this case the lumen of the needle 44 during deployment. The dimensions of the obturator 200 are such that the obturator 200 can be advanced through the lumen of the needle 44 and can exit the distal tip of the needle 44. The obturator 200 thus comprises an elongated structure 202, such as a solid rod or the like, that is advanced through the lumen of the needle 44. The diameter of the elongated structure 202 is thus slightly less than the diameter of the lumen of the needle 44 so that the obturator 200 is in close proximity or in contact with the inner wall of the needle 44 that defines the inner lumen.

The obturator 200 also includes a hub portion 210 to which the elongated structure 202 is coupled. The elongated structure 202 thus extends distally from the hub portion 210. The hub portion 210 is thus an enlarged part compared to the elongated structure 202. The elongated structure 202 terminates in a distal blunt end 205. In this embodiment, the obturator 200 can be a solid structure.

In the embodiment shown in FIGS. 2-5, the obturator 200 is configured to allow for the blood flash to occur in that in pre-deployment (initial) position in that the obturator 200 does not prevent blood from flowing through the needle into the flash chamber in the needle hub portion 42. In one embodiment, in the pre-deployment position, the blunt end 205 is spaced from the proximal end of the needle 44, thereby allowing the blood to flow through the needle 44 into the flash chamber in the needle hub 42. Once the obturator 200 is actuated by use of an actuator 210, the obturator 200 moves from the pre-deployment position to a deployed position in which the blunt end 205 is advanced distally beyond the sharp distal end of the needle 44 so as transform the sharp end of the needle 44 into a blunt end. In this deployed position, the wider proximal end (defined by the hub portion 210) contacts the inner shoulder 45. This inner shoulder 45 thus acts as a stop for forward advancement (firing) of the obturator 200.

The obturator 200 is a biased structure in that a biasing element 220, such as a spring, is provided and is disposed within the hub portion 42 adjacent the obturator 200. Spring 220 can be located between the proximal end of the obturator 200 and a mounting surface located internally within the needle hub 42. In the pre-deployment position, the spring 220 is in a compressed state and stores energy. An actuator 230 is provided and is operatively coupled to the obturator 200 such that when the actuator 230 is manipulated, the obturator 200 is released and the energy that is released from the spring 220 causes forward advancement (firing) of the obturator 200 within the needle lumen to the deployed position. The actuator 230 can take any number of different forms including a switch, button, slider, etc. The actuator 230 can have a catch or claw or the like that engages the obturator 200.

### Operation of IV Catheter Device 10 (FIGS. 2-5)

FIG. 2 shows the IV catheter device 100 is a pre-deployment position in which the needle 44 is exposed beyond the IV catheter component 20 and ready for insertion into the vein 5. Applicant notes that the angle between the needle 44 and vein 5 has been exaggerated for purpose of illustration and as mentioned herein, the needle 44 typically inserted into the vein at a minimum angle (as close to parallel to the skin). In FIG. 2, the obturator 200 is in the pre-deployment position and the biasing element (spring) 220 stores energy. Similarly, the IV catheter component 20 has not been advanced into the vein 5 and instead remains external to the body.

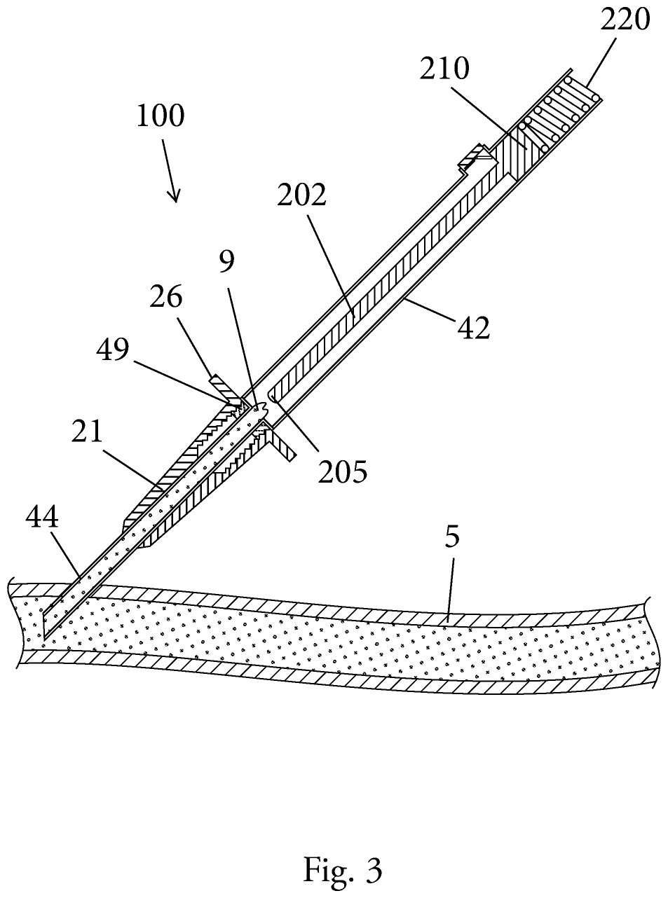

FIG. 3 shows the step of inserting the sharp end of the needle 44 into the vein 5. As discussed previously, once the needle 44 enters the vein 5, a flash of blood, generally indicated at 9, is seen. This blood flash occurs when blood flows into and up the needle 44 into a flash chamber in the hub portion 42 of the needle 40. The obturator 200 is still in the pre-deployment position and the IV catheter component 20 is outside the patient's body.

FIG. 4 shows the deployment of the obturator 200 by manipulation of the actuator 230. Once the actuator 230 is manipulated, the obturator 200 is released and the stored energy in the biasing element 220 is released. This release of energy causes the obturator 200 to be driven towards the vein 5. The obturator 200 is driven into and travels through and exits the sharp tip of the needle 44. The travel of the obturator 200 is terminated when the hub portion 210 contacts the shoulder 45 which acts as a stop. In this deployed position, the blunt distal end 205 of the obturator 200 extends distally beyond the sharp tip of the needle 44 to transform the sharp tip into a blunt end. However, the advancement of the obturator 200 is configured so that the blunt end 205 does not advance too far ahead of the sharp tip of the needle 44 since the blunt end 205 is not intended and should not contact the back wall of the vein 5.

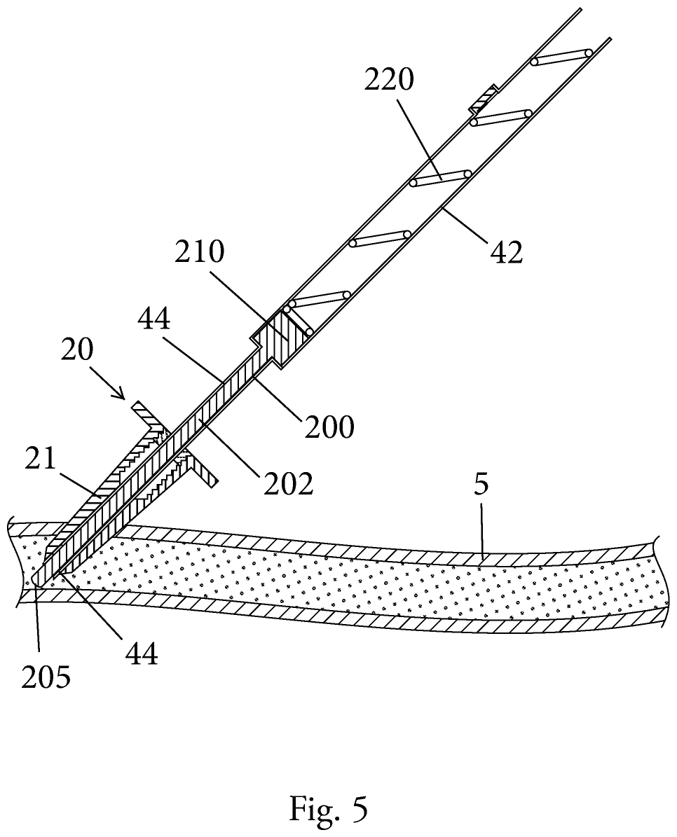

FIG. 5 shows the advancement of the distal end of the IV catheter body 21 into the vein 5. This action occurs manually by grasping the catheter body 21 and then advancing it forward over the needle 44 until the catheter tip (distal tip) is located within the vein 5. The blunt end 205 of the obturator 200 remains within the vein 5. Next, once the catheter tip is located within the vein 5, the needle 44 is removed from the IV catheter component 20. Since the obturator 200 is coupled to the needle component 40, the obturator 200 is removed as well.

The present invention thus provides a solution to high failure rates of IV catheter placement in the form of the spring-loaded, actuatable (e.g. button-released) obturator 200 which creates a blunt-tipped needle after needle tip is inside vessel (vein) 5. This would allow for a practitioner to safely advance needle 44 forward until catheter tip is also within vessel lumen without fearing penetration of needle through the posterior wall of the vessel (vein 5). This would likely be most beneficial for vessels which require steep angles of attack, such as ultrasound guided peripheral IVs placed in the obese patient. A secondary benefit of this system is that it would also reduce needle stick injuries as after removal, the needle tip would be blunt.

### Alternative Obturator Designs

Alternatively, in another embodiment, there is sufficient space surrounding the obturator 200 within the lumen of the needle 44 to permit the blood to flash by flowing around the obturator 200 to the flash chamber located in the wider hub portion 42 of the needle component 40. In this embodiment, in the pre-deployment position, the distal end portion of the elongated structure 202 of the obturator 200 is at least partially within the inner lumen of the needle 44. Since the diameter of the obturator 200 is less than the inner diameter of the needle 44, there is an annular space around the obturator 200. This annular shape opens into the flash chamber located in the wider hub portion 42. The wider hub portion 210 of the obturator is spaced away from the inner shoulder 45 in this pre-deployment position.

In the deployed position, the obturator 200 is advanced forward within the lumen of the needle 44 until the distal blunt end 205 is advanced distally beyond the sharp distal end of the needle 44 to once again transform the sharp needle tip into a blunt end.

### Hollow Obturator

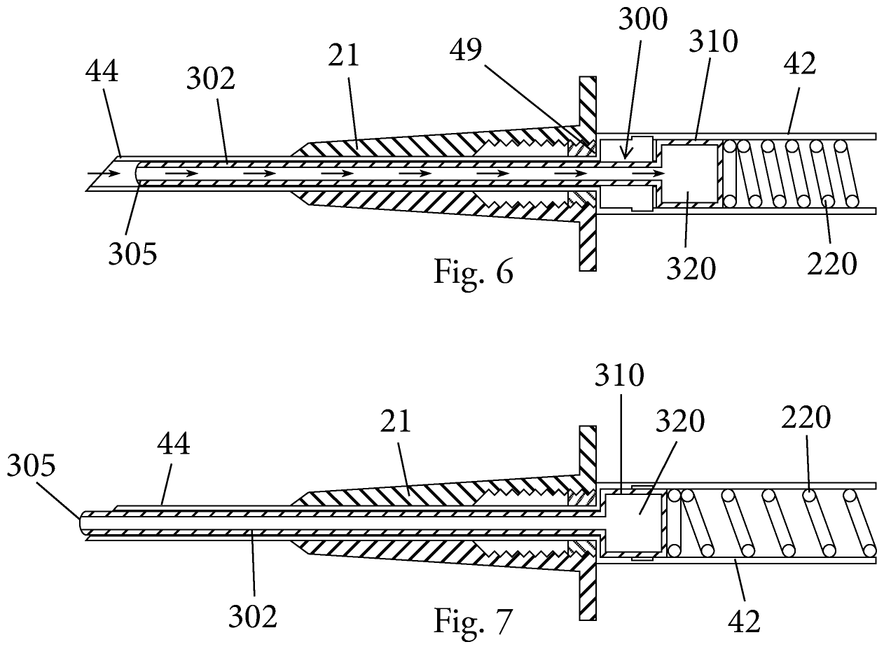

In another embodiment, the obturator is hollow as opposed to the solid obturator of the previous embodiments (See, FIG. 2). For example, in FIGS. 6-7, a hollow obturator 300 is provided. Like the obturator 200, the dimensions of the obturator 300 are such that the obturator 300 can be advanced through the lumen of the needle 44 and can exit the distal tip of the needle 44. The obturator 300 thus comprises an elongated hollow structure 302, such as a hollow rod or the like, that is advanced through the lumen of the needle 44. The diameter of the elongated structure 302 is thus the same as or slightly less than the diameter of the lumen of the needle 44 so that the obturator 300 is in close proximity or in contact with the inner wall of the needle 44 that defines the inner lumen.

The obturator 300 also includes a hub portion 310 to which the elongated structure 302 is coupled. The elongated structure 302 thus extends distally from the hub portion 310. The hub portion 310 is thus an enlarged part compared to the elongated structure 302. The elongated structure 302 terminates in a distal blunt end 305. The hub portion 310 is a hollow structure and within the hub portion 310, there is a flash chamber 320 that receives the blood flash.

Unlike the previous embodiments, in this embodiment, it is the obturator 300 itself that defines the blood flash pathway. In the pre-deployment position, the distal end portion of the hollow elongated structure 302 of the obturator 300 is at least partially within the lumen of the needle 44. Since the diameter of the obturator 300 is the same or slightly less than the inner diameter of the lumen of the needle 44, the obturator 300 completely occupies the lumen of the needle 44 and the two are in intimate contact with one another. As a result, blood flowing into the lumen of the needle 44 flows directly into the lumen of the hollow obturator 300. The blood first flows within the hollow elongated structure 302 and then into the flash chamber 320 (indicated by arrows). The hub portion 310 is spaced away from the inner shoulder 45 in this pre-deployment position.

In the deployed position (FIG. 7), the obturator 300 is advanced forward within the lumen of the needle 44 until the distal blunt end 305 is advanced distally beyond the sharp distal end of the needle 44 to once again transform the sharp needle tip into a blunt end. It will be appreciated that the observance of the flash chamber 320 continuing to fill after advancement of the obturator 300 is indicative that the obturator tip/needle tip still being located in the vein.

### Alternative Obturator Designs

Additional obturator designs are possible for use with various complementary needle components so as to define different types of IV catheter devices.

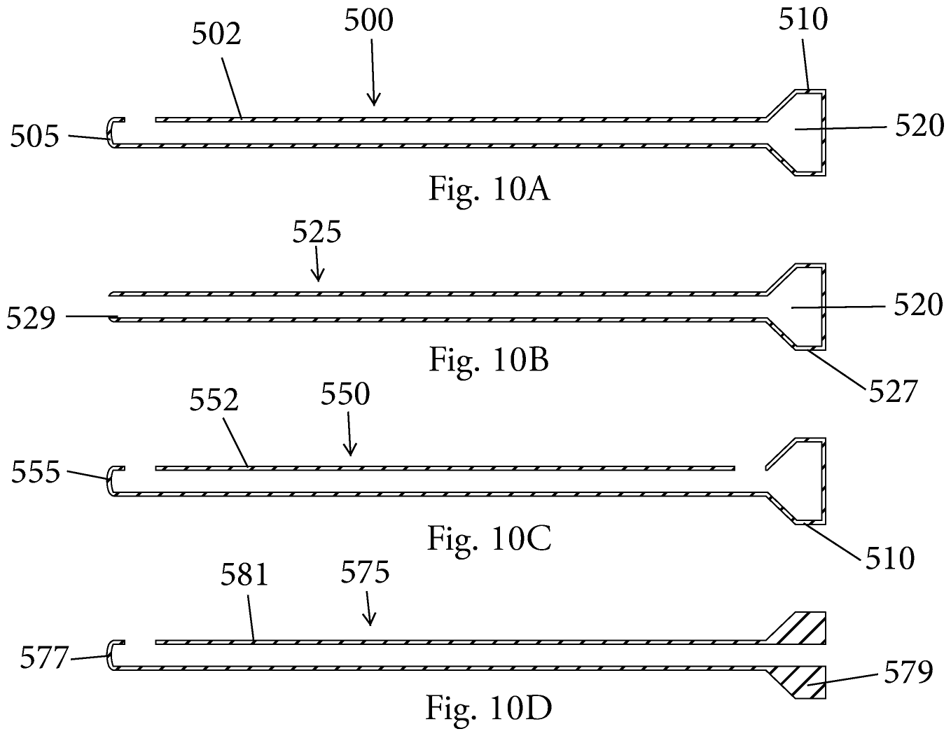

FIG. 10A shows an alternative obturator design 500. The obturator 500 is a hollow obturator like the obturator 300 and includes an elongated hollow structure 502 at one end and a hollow hub portion 510 at the other end. Unlike the previous embodiment, the inner lumen in the elongated structure 502 of the obturator 500 is not entirely coaxial and the distal end of the obturator 500 is not open but rather the entrance into the inner lumen of the obturator 500 is located along the side wall of the elongated structure 502 proximate the blunt distal end 505. Fluid (blood) can thus enter into the inner lumen and flow into a flash chamber 520 that is located within the hollow hub portion 510.

In this embodiment, the blunt end 505 is solid as a result of off-setting the entrance into the inner lumen of the elongated structure 502. This solid blunt end 505 extends beyond the beveled sharp end of the needle 44.

When the obturator 500 is advanced to its deployed position, the entrance to the inner lumen is positioned sufficiently beyond the sharp distal needle tip, thereby allowing blood from the vein 5 to flow into the open entrance into the inner lumen of the obturator 500. This feature and operation are described in more detail below.

FIG. 10B illustrates a hollow obturator design 525 that is similar to the hollow obturator shown in FIGS. 6 and 7. One difference being the shape of the hub portion 527 of the obturator 525. In FIG. 10B, the hub portion 527 has forward angled walls that are at an angle other than 90 degrees. The obturator 525 includes a blunt distal end 529 and has a flash chamber 520 defined within the hub portion 527. The entrance into the inner lumen is located at the distal end itself.

FIG. 10C shows an alternative obturator design 550. The obturator 550 is a hollow obturator like the obturators 300, 500 and includes an elongated hollow structure 552 at one end and a solid hub portion 510 at the other end. The inner lumen in the elongated structure 552 of the obturator 550 is not entirely coaxial and the distal end of the obturator 550 is not open but rather the entrance into the inner lumen of the obturator 550 is located along the side wall of the elongated structure 552 proximate the blunt distal end 555. The inner lumen of the obturator 550 has an exit that is also formed along the side wall of the elongated structure 552 proximal to the enlarged solid hollow hub portion 510. The entrance and the exit are thus located along the same side wall of the elongated structure 552.

In this embodiment, the inner lumen is thus a flow through channel in that blood enters into the inner lumen from the side of the obturator and flows through the inner lumen and then exits the obturator 550. From the exit, the blood flows radially outward into a flash chamber where it collects. The flash chamber is thus located outside the obturator 550 within the hub portion of the needle. When the obturator 550 is advanced, blood flows through the inner lumen of the obturator 550 into the flash chamber. In other words, when the obturator 550 is advanced to its deployed position, the entrance to the inner lumen is positioned sufficiently beyond the sharp distal needle tip, thereby allowing blood from the vein 5 to flow into the open entrance into the inner lumen of the obturator 550.

FIG. 10D shows yet another obturator 575 in which the inner lumen formed in the elongated structure 581 has an entrance along the side wall proximate the blunt end 577 and has an exit in the solid hub portion 579 at the proximal end of the obturator 575. Blood can thus enter from the side entrance once the distal blunt end 577 is located beyond the sharp tip of the needle in the deployed state. In this deployed state, the entrance to the inner lumen is open to the blood within the vein and the exit is in fluid communication with the flash chamber in the hub portion 42 of the needle component.

In this embodiment, the blunt end 577 is solid as a result of off-setting the entrance into the inner lumen of the elongated structure 581. This solid blunt end 577 extends beyond the beveled sharp end of the needle 44.

When each of the obturators 500, 550, 575 is advanced to its deployed position, the entrance to the inner lumen is positioned sufficiently beyond the sharp distal needle tip, thereby allowing blood from the vein 5 to flow into the open entrance into the inner lumen of the obturator. Thus, the inner lumen of the obturator remains open in the fully deployed position.

### Additional Needle and Obturator Designs

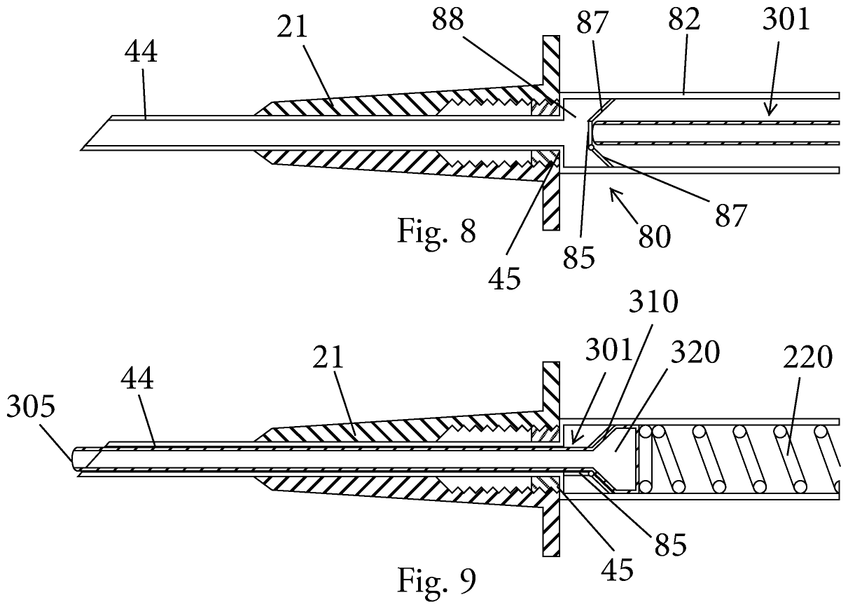

FIGS. 8 and 9 illustrate an obturator 301, that is similar to obturator 300, disposed within a needle component 80 that is similar to the needle component 40. A hub portion 82 of the needle component 80 has a first region in which the obturator 301 is disposed in the pre-deployment position. The obturator 300 includes a blunt end 305 with an inner lumen being formed within the obturator 300. The inner lumen extends to a flash chamber 320 formed in the obturator hub portion 310. This first region is defined by an inner wall that has a movable center portion 85 and an outer peripheral portion 87 that surrounds the center portion 85. Between the shoulder 45 and the inner wall there is an open space 88 that functions as the flash chamber formed in the needle hub portion 82.

The movable center portion 85 can be in the form of a hinged door that in a normal at rest position (when the obturator 301 is in the pre-deployment position) is closed. The door is hinged such that when a forward force is applied by the obturator 301, as it is fired forward, the door opens outward toward the shoulder 45. The door opens sufficiently such that the obturator 301 can advance forward and travel into the inner lumen of the needle 44 and exit the inner lumen as shown in FIG. 9 so as to transform the sharp end of the needle 44 into a blunt end.

The outer peripheral portion 87 can be angled as shown and can define a seat for the obturator hub. In particular, the obturator hub in the deployed position seats against the outer peripheral portion 87 which has a complementary shape relative to the shape of the obturator hub and acts as a stop. When the outer peripheral portion 87 is angled, the obturator hub can be angled and mirror the shape of the seat (stop).

The movable center portion 85 can be biased or constructed so that at rest it assumes the closed position and it takes an applied force, such as the driving action of the obturator, to move the movable center portion 85 to the open position.

The obturator hub thus defines the flash chamber 320 in this embodiment and is preferably transparent to allow the blood flash to be seen.

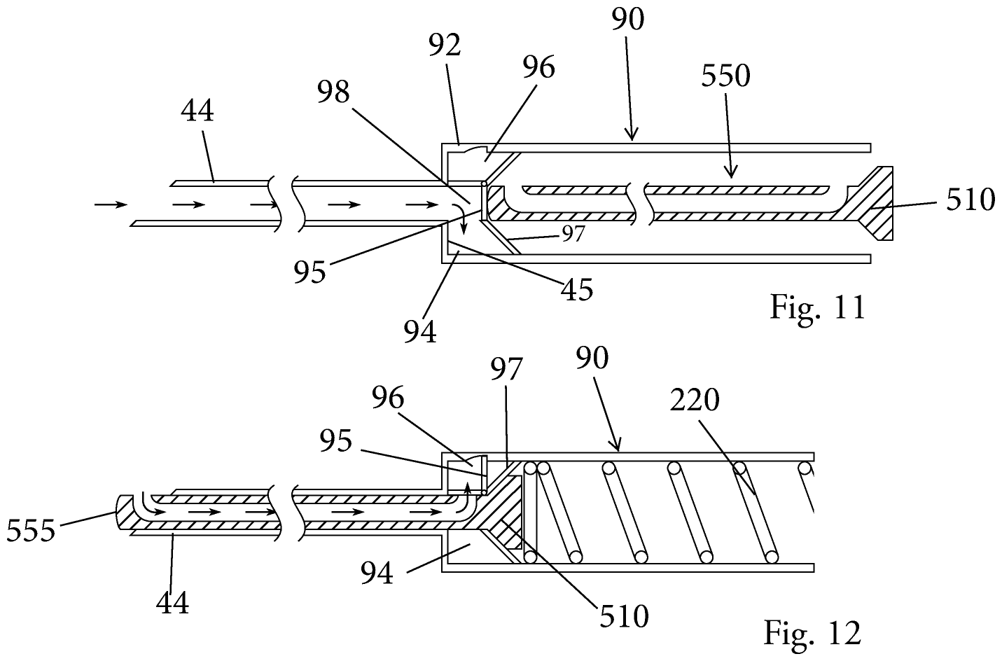

FIGS. 11 and 12 illustrate the obturator 550 (FIG. 10C) disposed within a needle component 90 that is similar to the needle component 40. A hub portion 92 of the needle component 90 has a first region in which the obturator 550 is disposed in the pre-deployment position. This first region is defined by an inner wall that has a movable center portion 95 and an outer peripheral portion 97 that surrounds the center portion 95. Between the shoulder 45 and the inner wall there is an open space 98 that functions as the flash chamber.

In this particular embodiment, the open space 98 defines two different flash chambers, namely, a first flash chamber 94 that receives a first blood flash and a second flash chamber 96 that receives a second blood flash. The second flash chamber 96 is closed off in the pre-deployment position of the obturator 550, while the first flash chamber 94 remain open.

Thus, when the needle 44 enters the vein (vessel), the first blood flash travels up the inner lumen of the needle and is directed by the inner wall into the first flash chamber 94. The second flash chamber 96 remains closed and as a result, the blood flash is directed into the first flash chamber 94.

The movable center portion 95 can be in the form of a hinged door that in a normal at rest position (when the obturator 550 is in the pre-deployment position) is closed. The door is hinged such that when a forward force is applied by the obturator 550, as it is fired forward, the door opens outward toward the shoulder 45. The door opens sufficiently such that the obturator 550 can advance forward and travel into the inner lumen of the needle 44 and exit the inner lumen as shown in FIG. 12 so as to transform the sharp end of the needle 44 into a blunt end.

The outer peripheral portion 97 can be angled as shown and can define a seat for the obturator hub. In particular, the obturator hub in the deployed position seats against the outer peripheral portion 97 which has a complementary shape relative to the shape of the obturator hub and acts as a stop. When the outer peripheral portion 97 is angled, the obturator hub can be angled and mirror the shape of the seat (stop).

Once the obturator 550 is fired, the second flash chamber 96 opens up due to the pivoting of the movable center portion 95 as well as the fluid force applied by the blood which can, as shown in FIGS. 11 and 12, cause further pivoting of the center portion 95. At the same time, the body of the obturator closes off the first flash chamber 94. Due to the construction of the inner lumen formed within the obturator, blood flash after advancement of the obturator 550 is permitted since the entrance into the inner lumen formed in the obturator is exposed beyond the distal end of the needle and the exit of the inner lumen of the obturator axially aligns with the now open second flash chamber 96 and therefore, blood is directed into this second flash chamber 96. The first and second flash chambers 94, 96 can be separated from one another by a wall or the like. Alternatively, the first and second flash chambers 94, 96 can be in fluid communication with one another.

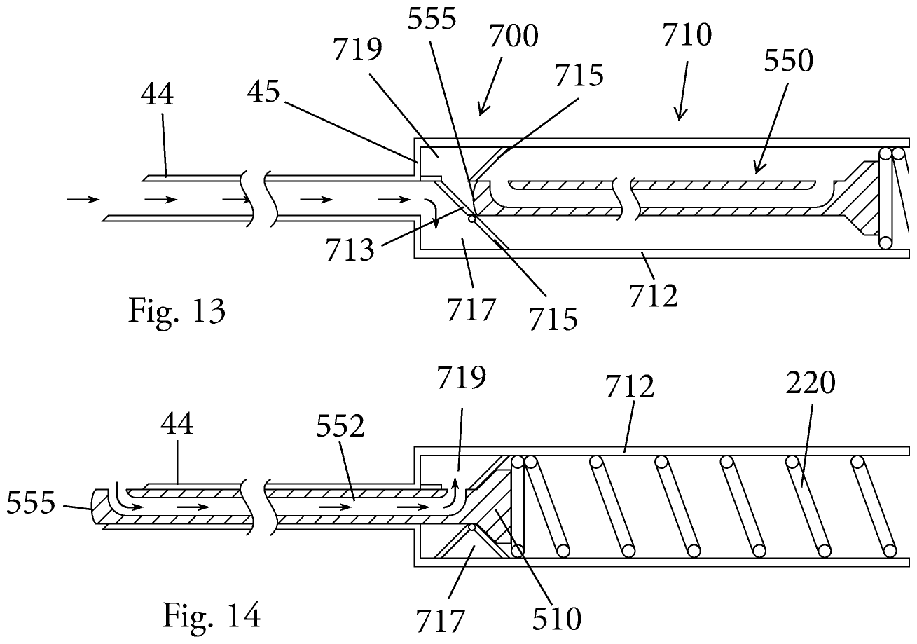

FIGS. 13 and 14 illustrate another IV catheter device 700 that includes a needle component 710 that includes needle 44 and hollow hub portion 712. Within the hub portion 712, there is an inner wall that includes a movable center portion 713 and a peripheral portion 715. The movable center portion 713 moves between a closed position (FIG. 13) and an open position (FIG. 14). An open space is formed between the shoulder 45 and the inner wall and more specifically, the open space includes a first flash chamber 717 and a second flash chamber 719. As shown in FIG. 13, in the pre-deployment position, the movable center portion 713, which can be in the form of hinged door, closes off the second flash chamber 719, while the first flash chamber 717 remains open. The obturator 550 remains behind the inner wall in the pre-deployment position.

When the needle 44 is first inserted into the vein, the first blood flash flows into and with the inner lumen of the needle 44 and flows into the first flash chamber 717 but not the second flash chamber 719.

In the deployed position of FIG. 14, once the obturator 550 is fired forward, the obturator 550 contacts the movable center portion 713 and causes it to swing open to allow for passage of the obturator 550 into the inner lumen of the needle 44. The blunt distal end 555 of the obturator is located outside the sharp distal end of the needle 44 so as to transform it into a blunt end. The side entrance into the inner lumen of the obturator 550 is located beyond the sharp distal end of the needle 44 to allow for blood flow into the inner lumen formed in the elongated structure 552 of the obturator 550. The side exit of the inner lumen near the hub portion 510 is axially aligned with the second flash chamber 719 to allow the blood to flow from the inner lumen into the second flash chamber 719 (in its pivoted state). The first flash chamber 717 can be closed off by the movable center portion 713 as well as by the body of the obturator.

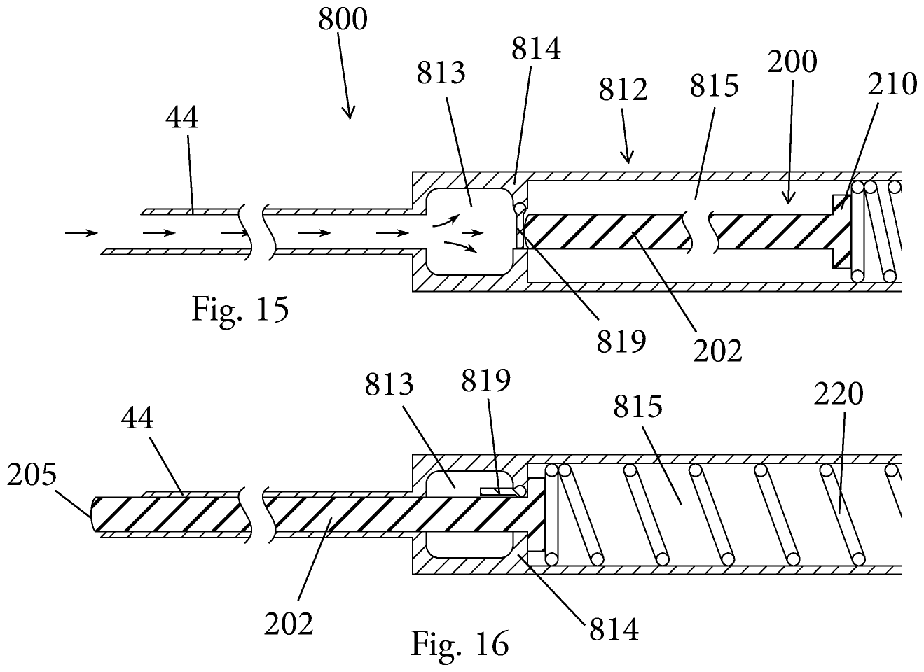

FIGS. 15 and 16 illustrate another IV catheter device 800 that includes a needle component 810 that includes needle 44 and hollow hub portion 812. Within the hub portion 812, there is an inner wall 814 that partitions the hub portion 812 into a forward compartment 813 and a rear compartment 815. The forward compartment 813 is in fluid communication with the inner lumen of the needle 44. Between the forward compartment 813 and the rear compartment 815, there is an openable door 819 that moves between the closed position of FIG. 15 and the open position of FIG. 16. The openable door 819 can be a hinged door and is sized so that the elongated structure 202 of the obturator 200 can pass therethrough. The inner wall 814 acts as a stop for the forward advancement of the obturator 200 as described below.

In the pre-deployment position of FIG. 15, the obturator 200 is disposed within the rear compartment 815 along with the spring 220. When the needle 44 pierces the vein wall, the blood flash flows up into the forward compartment 813 that comprises a flash chamber.

Once the needle 44 is in the proper position, the obturator 200 is fired forward and this action causes the obturator 200 to strike and force open the hinged door 819 to allow the elongated structure 202 to travel into the needle 44. As with the other embodiments, in the deployed position, the distal blunt end 205 of the obturator 200 is advanced beyond the sharp tip of the needle 44 and the hub portion 210 of the obturator 200 strikes and is biased against the inner wall 814 which acts a stop as shown in FIG. 16. The obturator 200 thus passes through the forward compartment 813. However, the obturator does not fill the entire forward compartment 813 and flash blood is accommodated.

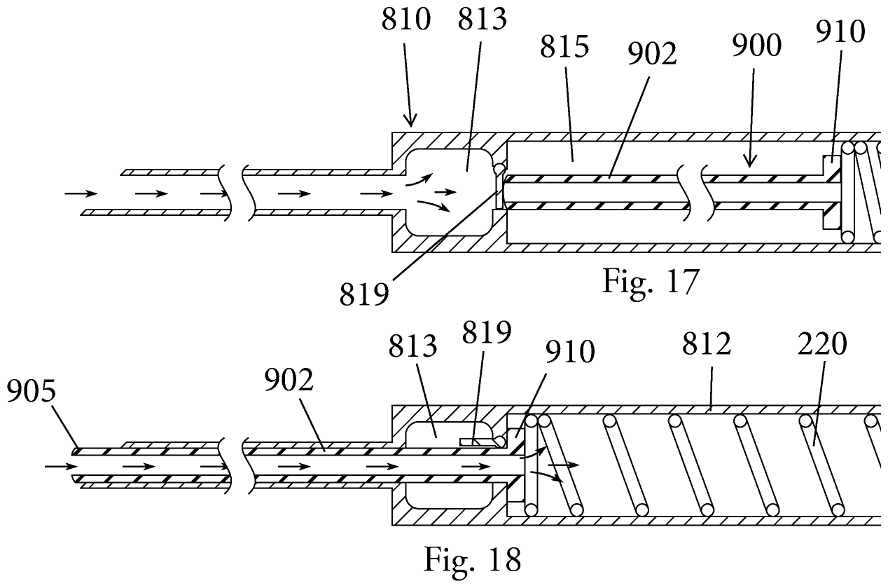

FIGS. 17 and 18 show another embodiment in which the needle component 810 is used with an obturator 900 that is similar to the one shown in FIG. 10D. The obturator 900 is a hollow obturator like the obturators described above and includes an elongated hollow structure 902 at one end and a hub portion 910 at the other end. The inner lumen in the elongated structure 902 of the obturator 900 is formed along one axis. The inner lumen is thus open at the blunt distal end 905 and extends completely through the obturator and has an exit at the hub portion 910. The blood thus flows axially and longitudinally through the body of the obturator 900 to a flash chamber formed in the rear compartment 815 of the hub portion of the needle component 810.

In the pre-deployment position of FIG. 17, the obturator 900 is disposed within the rear compartment 815 along with the spring 220. When the needle 44 pierces the vein wall, the blood flash flows up into the forward compartment 813 that comprises an initial flash chamber.

Once the needle 44 is in the proper position, the obturator 900 is fired forward and this action causes the obturator 900 to strike and force open the hinged door 819 to allow the elongated structure 902 to travel into the needle 44. As with the other embodiments, in the deployed position, the distal blunt end 905 of the obturator 200 is advanced beyond the sharp tip of the needle 44 and the hub portion 910 of the obturator 900 strikes and is biased against the inner wall 814 which acts a stop as shown in FIG. 18. The obturator 900 thus passes through the forward compartment 813.

The hollow obturator 900 design thus accommodates blood flow through the obturator 900 in the deployed position. Blood can flow into and through the inner lumen of the obturator 900 and then exits through the exit in the hub portion 910 and into the second compartment 815 which acts as a second flash chamber. Since the hub portion of the needle is preferably transparent, this second blood flash will be visible.

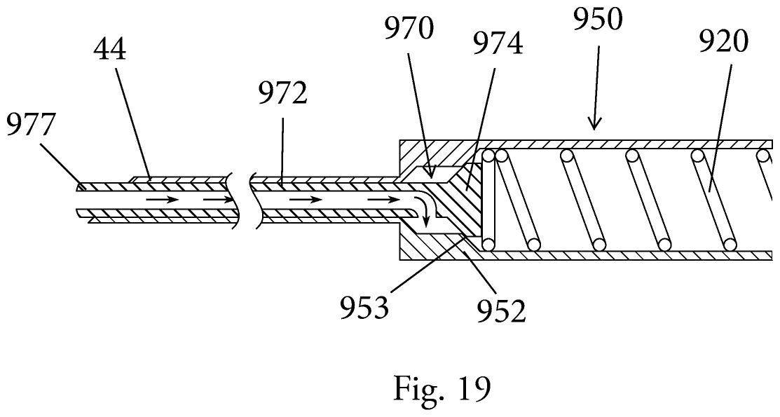

FIG. 19 sets forth another embodiment in which a needle component 950 is used with an obturator 970. The obturator 970 is a hollow obturator like the obturators described above and includes an elongated hollow structure 972 at one end and a hub portion 974 at the other end. The inner lumen in the elongated structure 972 of the obturator 970 is open at the blunt distal end 977 and has an exit along a side wall of the elongated structure 972 near the hub portion 974.

The needle component 950 includes needle 44 and a hollow hub portion 952. Within the hollow hub portion 952 has an inner wall 953 that acts as a stop. The inner wall 953 can be an angled wall.

In the deployed position, the blood thus flows axially and longitudinally through the inner lumen of the obturator 970 to a flash chamber formed in the hub portion 952 of the needle component 950. The hollow obturator 970 design thus accommodates blood flow through the obturator 970 in the deployed position. Biasing element (spring) 920 is provided.

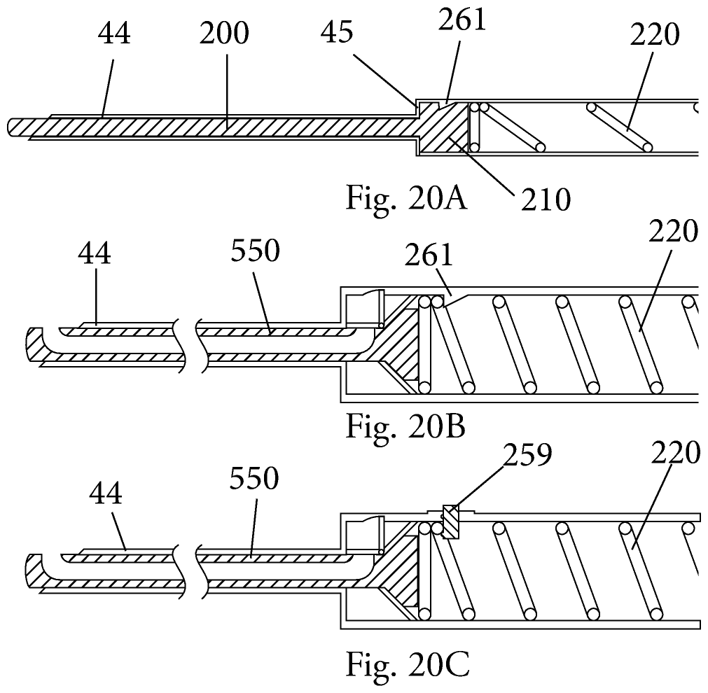

### Lock or Retaining Mechanism (FIGS. 20A-20C)

It will be appreciated that after deployment of the actuatable obturator 200, a mechanism can be provided to ensure that the actuatable obturator remains in the deployed position and does not back into the inner lumen of the needle after deployment. If the actuatable obturator 200 backs into the inner lumen of the needle, the sharp tip would be exposed again.

To ensure that the actuatable obturator 200 remains in the deployed position several techniques can be used.

One technique is the use of a lock mechanism or the like which itself is operable after the actuatable obturator 200 is deployed as shown in FIG. 20C. One type of lock mechanism can be manually operated after deployment of the actuatable obturator 200 as by the user pressing a button 259 or the like that operates the locking mechanism. For example, a button or the like that is located along the side wall of the hub portion of the needle can be operated by the user and be caused to engage the hub portion of the actuatable obturator 200. For example, a locking tab or pin can be driven into position along the proximal face of the hub portion of the actuatable obturator 200, thereby preventing unintended retraction of the actuatable obturator 200 into the inner lumen of the needle. Alternatively, the locking tab or pin can be driven into contact with a forward part of the spring element 220, thereby preventing the spring element 220 from compressing. This arrangement thus restricts rearward movement of the obturator since the locking tab or pin acts as a stop or obstruction.

This type of lock mechanism can be configured so that the user can reverse and release the lock mechanism, thereby freeing the actuatable obturator. This would allow the manual retracting of the actuatable obturator 200 to purposefully recreate the sharp tip, but the retraction should not be from pressure applied at the forward tip.

In addition, the lock mechanism can be automated and tripped by the deployment of the actuatable obturator 200. For example, the enlarged hub portion of the actuatable obturator 200 can contact and trip a locking mechanism that then engages the hub portion, such as the proximal end of the hub portion, to create a mechanical interference that prevents retraction of the actuatable obturator within the inner lumen of the needle (i.in the proximal direction).

FIG. 20A shows a locking tab 261 that is formed along the inner wall of the hub portion of the needle. The tab 261 is able to flex slightly outward to accommodate the forward movement of the obturator 200. In particular, when the leading edge of the hub portion 210 of the obturator contacts the locking tab 261, this force causes the locking tab 261 to be flexed outward until a complementary notch (cut out) formed in the hub portion 210 aligns with the locking tab 261 at which time the locking tab 261 flexes inward and is received within the complementary notch in the hub portion 210. This reception and engagement of the locking tab 261 within the complementary notch of the hub portion 210 occurs when the hub portion 210 seats against the forward stop (shoulder 45). The locking tab 261 has a ramp construction and a forward edge of the locking tab 261 is a right angle shoulder that is oriented perpendicular to the inner wall of the hub portion of the needle.

FIG. 20C is similar to FIG. 20B but in this embodiment, the locking tab 261 engages the windings of the forward section of the spring element 220. The hub portion of the obturator 550 is sized to pass by the locking tab 261 without contact or engagement therewith. When the obturator 550 is fired forward, the forward end of the spring element 220 rides up the ramp of the locking tab and once the spring element 220 clears the locking tab 261, the spring element 220 extends radially outward to its normal position. However, the forward edge of the locking tab 261 which is in the form of a right angle shoulder prevents the spring from moving rearward and being compressed. This arrangement thus restricts rearward movement of the obturator since the locking tab or pin acts as a stop or obstruction.

Alternatively, the spring element 220 has a selected high compressive strength that prevents unintended retraction of the actuatable obturator 200 within the inner lumen of the needle during normal use of the device within the vessel (vein).

It will also be understood that the obturators shown in FIGS. 20A-20C are only exemplary in nature and the lock mechanisms shown in FIGS. 20A-20C can be used with any of the other obturators shown herein so long as the arrangement is operative. Thus, each lock mechanism is generally configured to prevent unintended retraction of the obturator after it has been deployed.

In addition, it will be understood that the lock mechanism illustrated in the figures and described herein can be implemented in all of the embodiments described and illustrated herein. In other words, while the figures do not show the lock mechanism incorporated into each of the illustrated embodiments, each of these embodiments preferably includes a lock mechanism to ensure that the deployed obturator does not freely move rearward when a force is applied to the blunt distal end.

### Echogenic Obturator 400

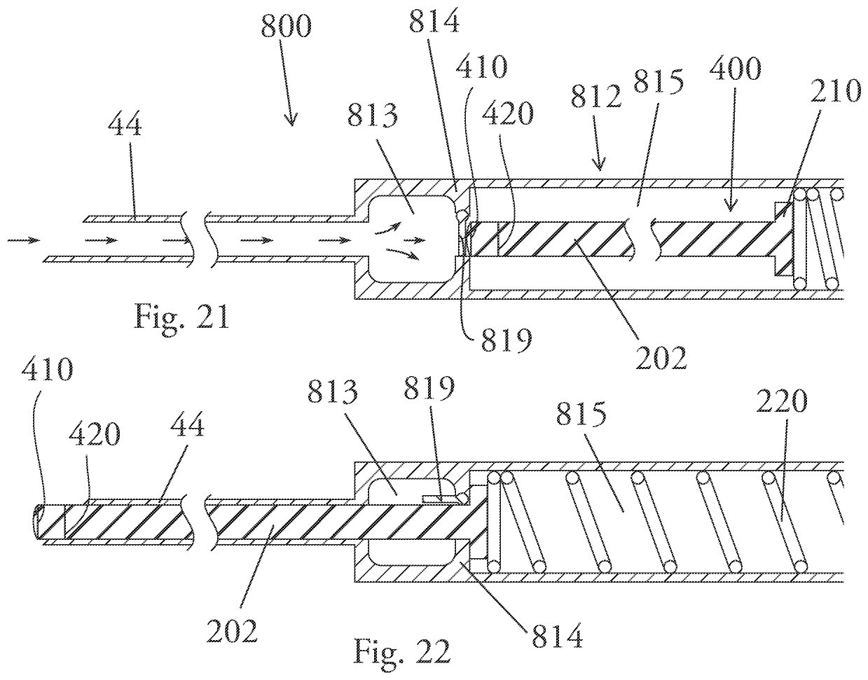

In another aspect of the present disclosure shown in FIGS. 21-23, the devices disclosed therein can include an echogenic obturator 400. The obturator 400 is shown as being part of the device 800 which has been previously described in detail with respect to other embodiments. Thus, all of the parts and features of 800 are not described again but instead, like elements have like reference numbers. As is known, echogenicity is the ability to bounce an echo (e.g., return signal) in ultrasound examinations.

It will also be appreciated that the teachings illustrated in the obturator 400 of FIGS. 21-23 can be implemented in the other devices described herein and illustrated in the other figures. In other words, the teachings with respect to FIGS. 21-23 are not limited to the devices shown therein but instead, the obturator 400 can used in the other devices.

The obturator 400 contains design features which allow for increased echogenicity and improved visualization when placed under ultrasound guidance. The features could contain either materials which are inherently more echogenic than the surrounding materials or entail etchings in the obturator which increase the number of ultrasound beams returning to the probe. This increased echogenicity would be used after deployment of the obturator 400 upon obtaining a flash of blood. The improved visualization of the obturator 400 would provide additional information about how far and at what orientation to advance the system into the vessel. The features may be placed at predetermined intervals to aid in determining how far into the vessel the user has advanced.

FIG. 21 contains one or more regions of echodense material that provide increased echogenicity. In FIG. 21, the echodense regions are provided in the form of one or more echodense hands. In particular, at or proximate the distal end (distal tip) of the obturator 400, there can be at least a first band 410 and a second band 420 that is longitudinally spaced from the first band 410. Each of the first band 410 and the second band 420 can comprise an etched region of the obturator or can be formed of an echodense material. The first band 410 and the second band 420 can be in the form of continuous etched rings that extend circumferentially about the outer surface of the obturator body. In one embodiment, the first band 410 and the second band 420 can be etchings in the obturator body that are at predetermined interval distances. These etchings provide increased echogenicity of the obturator 400 after deployment as shown in FIG. 22. In FIG. 22, the obturator 400 has been deployed and at least the distal tip extends distally beyond the needle tip. The result of the etchings can be the formation of peaks and valleys along the outer surface.

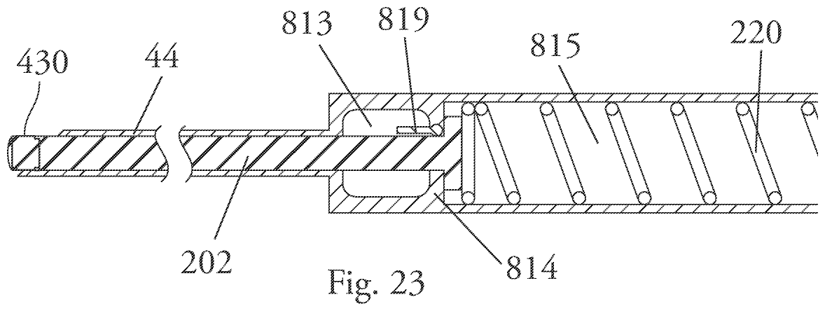

FIG. 23 illustrates an alternative design in which instead of one or more spaced etchings, the obturator includes a solid echodense tip 430. It will also be understood that the entire obturator 400 can be formed of an echodense material that is different than the other material.

One skilled in the art would understand that there are many different types of materials that are echodense to provide visualization under traditional imaging techniques (e.g. ultrasound). Etchings provide different scattering patterns to allow for improved visualization.

As mentioned herein, the provision of one or more regions of echodense material provide for increased visualization of the obturator especially post deployment. When more than one region of echodense material is provided at spaced intervals, the degree of deployment can be determined. For example, as shown in FIGS. 21-22, the user can determine the degree of obturator deployment by visualization of the locations of the one or more echodense regions relative to other landmarks. For example, the width of the bands 410, 420 are known and therefore, the degree of which the band is visible beyond the needle tip allows the position of the obturator 400 to be easily determined.

While the applications of the present catheter devices are discussed in terms of being used in intravenous access, it will be appreciated that the present devices can be used in other applications. For example, the present devices can be used in other kinds of vascular access (intra-arterial) or other procedures where it might be useful to move from a sharp to a blunt tip, such as peripheral nerve blocks or neuraxial procedures.

In all of the embodiments described herein, the obturator can be formed of a transparent material to allow the user to see the blood flash. In addition, the hollow hub portion of the needle component is also typically transparent.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presences of stated features, integers, steps, operations, elements, and/or components, but do not precludes the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

1. An IV catheter device comprising:
a needle component having an elongated needle with a sharp distal tip and a hollow needle hub from which the elongated needle extends, the hollow needle hub defining a flash chamber;
a catheter component having a hollow body through which the needle passes; and
an actuatable obturator that is disposed within the needle component and moves between a pre-deployment position and a deployed position, wherein in the deployed position, the actuatable obturator is disposed within an inner lumen of the elongated needle and a blunt distal end of the obturator is located distal to the sharp distal tip so as to blunt the elongated needle;
wherein the actuatable obturator is biased by a biasing element that stores energy in the pre-deployment position and releases energy as the actuatable obturator moves from the pre-deployment position to the deployed position; the biasing element being disposed within the hollow needle hub within the flash chamber defined in the hollow needle hub;
wherein the actuatable obturator is hollow and has an inner lumen formed therein that is open at proximal and distal ends of the obturator, the inner lumen at the proximal end of the obturator being open to an area of the flash chamber that contains the biasing element.

2. (canceled)

3. The IV catheter device of claim 1, wherein the biasing element comprises a coil spring and the inner lumen is axially aligned with a center of the coil spring.

4. The IV catheter device of claim 1, wherein the actuatable obturator includes an elongated section that includes the blunt distal end of the actuatable obturator and an enlarged hub portion that is wider than the elongated section of the actuatable obturator.

5. The IV catheter device of claim 4, wherein the needle component includes an internal stop and in the deployed position, the hub portion of the actuatable obturator seats against the stop.

6. The IV catheter device of claim 5, wherein the stop comprises an inner shoulder formed in the hollow hub portion of the needle component, the elongated needle extending distal to the hollow hub portion.

7. The IV catheter device of claim 6, wherein a length of the elongated section is selected so that when the hub portion of the actuatable obturator seats against the stop, the blunt distal end of the obturator is located distal to the sharp distal tip.

8. (canceled)

9-11. (canceled)

12. The IV catheter device of claim 1, wherein the actuatable obturator includes an actuator disposed along the hollow hub portion of the needle component, the actuator being operatively coupled to the actuatable obturator such that activation of the actuator releases the actuatable obturator allowing the biasing element to advance the actuatable obturator forward toward the sharp distal tip of the needle.

13. The IV catheter device of claim 1, wherein the hollow hub portion of the needle component includes an inner wall that partitions a hollow interior of the hollow hub portion into a forward section and a rear section, wherein in the pre-deployment position, the actuatable obturator is contained completely within the rear section, the inner wall including a movable door portion that moves between a closed position when the actuatable obturator is in the pre-deployment position and an open position when the actuatable obturator is in the deployed position, wherein the forward section comprises a flash chamber.

14. The IV catheter device of claim 13, wherein the movable door comprises a hinged door that naturally assumes the closed position in a rest state of the pre-deployment position.

15. The IV catheter device of claim 14, wherein the hinged door is centrally located along the inner wall and the blunt distal end of the obturator contacts one side of the hinged door in the pre-deployment position.

16. The IV catheter device of claim 14, wherein a width of the hinged door is substantially equal to a width of an elongated structure of the obturator.

17-26. (canceled)

27. An IV catheter device comprising:
a needle component having an elongated needle that extends outwardly from a needle hub, the needle hub having a distal end wall;
a catheter component that is detachably coupled to the needle component, the catheter component having a hollow body through which the elongated needle passes, the catheter component having a flange at a proximal end; and
an actuatable obturator that is slidingly disposed within the needle component and moves between a pre-deployment position and a deployed position, the actuatable obturator having an elongated hollow structure that terminates in an open blunt distal end and a hollow hub portion that is enlarged relative to the elongated hollow structure and defines a flash chamber, the hollow hub portion defining a proximal end of the actuatable obturator and including a distal end wall;
wherein in both the pre-deployment and deployed positions, the entire hollow hub portion of the actuatable obturator is fully contained within the needle hub;
wherein in the deployed position, the actuatable obturator is disposed within an inner lumen of the elongated needle and a blunt distal end of the obturator is located distal to the sharp distal tip so as to blunt the elongated needle;
wherein the obturator has an inner lumen formed therein with an entrance to the inner lumen of the obturator being open and distal to the sharp distal tip of the elongated needle when the obturator is in the deployed position.

28. (canceled)

29. The IV catheter device of claim 27, wherein in the pre-deployment position, the distal end wall of the needle hub seats against the flange of the catheter component and the distal end wall of the actuatable obturator is spaced from the distal end wall of the needle hub; and
wherein in the deployed position the distal end wall of the hub portion of the actuatable obturator seats against the distal end wall of the needle hub.

30. The IV catheter device of claim 27, wherein a side surface of the hollow hub portion of the actuatable obturator seats directly against an inner surface of the needle hub.

31. The IV catheter device of claim 27, further including a spring contained within the needle hub and seating at one end against a proximal end of the hollow hub portion of the actuatable obturator for applying a biasing force against the actuatable obturator.

32. The IV catheter device of claim 31, further including a movable stop against which the distal end wall of the hollow hub portion of the actuatable obturator seats in the pre-deployment position, wherein in the deployed position, the movable stop is displaced from a location in front of the distal end wall of the hollow hub portion of the actuatable obturator.

33. The IV catheter device of claim 32, wherein the inner surface of the needle hub includes a recess for receiving the movable stop in the deployed position.

34. An IV catheter device comprising:
a needle component having an elongated needle that extends outwardly from a needle hub;
a catheter component that is detachably coupled to the needle component, the catheter component having a hollow body through which the elongated needle passes, the catheter component having a flange at a proximal end; and
an actuatable echogenic obturator that is disposed within the needle component and moves between a pre-deployment position and a deployed position, wherein the actuatable echogenic obturator includes at least one echogenic element that provides localized increased echogenicity of the actuatable echogenic obturator and improved visualization of the actuatable echogenic obturator, wherein the actuatable echogenic obturator includes at least one region that is adjacent the at least one echogenic element and does not provide increased echogenicity;
wherein in the deployed position, the actuatable echogenic obturator is disposed within an inner lumen of the elongated needle and a blunt distal end of the actuatable echogenic obturator is located distal to a sharp distal tip of the elongated needle so as to blunt the elongated needle.

35. The IV catheter device of claim 34, wherein the at least one echogenic element is located at the blunt distal end.

36. The IV catheter device of claim 34, wherein the at least one echogenic element comprises a first band and a second band spaced longitudinally from the first band.

37. The IV catheter device of claim 36, wherein each of the first band and the second band comprises one of an etched region of the actuatable echogenic obturator and a region that is formed of an echodense material.

38. The IV catheter device of claim 1, wherein in the fully deployed position, an entire width of a least a section of the at least one echogenic element is visible beyond the sharp distal tip of the elongated needle.
